(19) United States
(12) Patent Application Publication
(10) Pub. No.: US 2025/0295884 A1
(43) Pub. Date: Sep. 25, 2025

(54) BREATH BEADS

(71) Applicant: MACEY ERIN HICKOX, COLORADO SPRINGS, CO (US)

(72) Inventor: MACEY ERIN HICKOX, COLORADO SPRINGS, CO (US)

(21) Appl. No.: 18/613,751

(22) Filed: Mar. 22, 2024

(51) Int. Cl. A61M 21/02; A61M 21/00

(52) U.S. Cl. CPC A61M 21/02; A61M 2021/0022

(57) ABSTRACT

The invention of Breath Beads utilizes a simple tool that is regularly on a person, often, and when used with a little education functions as a piece of equipment for breathing exercises and aid for meditation practices. The initial product uses a bracelet with 30 beads to represent 30 breaths performed at a slow rate of 6 breaths per minute for a 5 minute practice. Beads at the minute markers and finish of the bracelet have varied texture, size and/or color to serve as a tactile reference of breath count and time of practice. A healthy, slow breathing rate is encouraged to be paired with a meditation practice to experience holistic physical, emotional, mental and spiritual health.

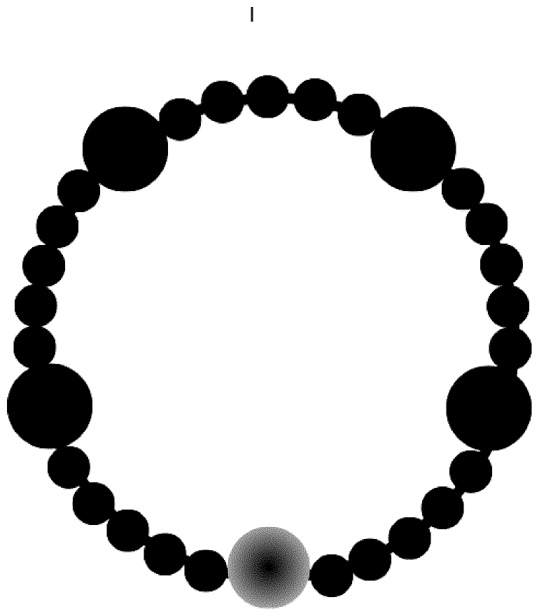

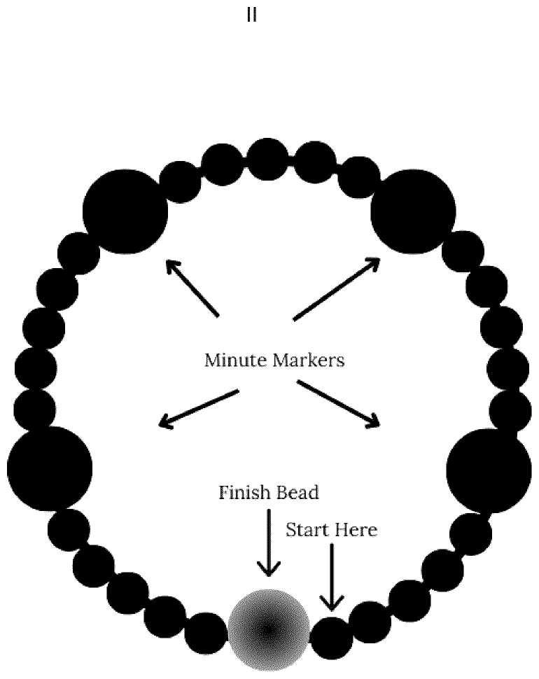

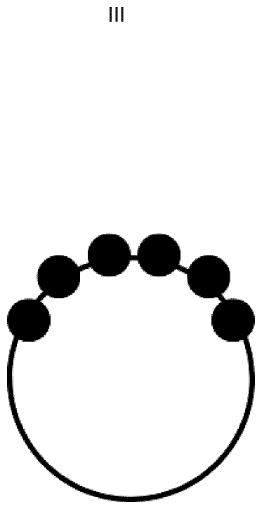

### CROSS-REFERENCE NONPROVISIONAL DESIGN PATENT

Application No. 29/922,090. Confirmation No. 6318. Filing Date: Dec. 20, 2023

### STATEMENT REGARDING FEDERALLY SPONSORED RESEARCH OR DEVELOPMENT

Not applicable

### REFERENCE TO A SEQUENCE LISTING, A LARGE TABLE, OR COMPUTER PROGRAM LISTING APPENDIX ON READ-ONLY OPTICAL DISC

Not applicable

### BACKGROUND OF THE INVENTION

The Breath Beads invention is using a (product) bracelet with beads/markers to represent breath count and functions as a respiratory timer. Breath Beads are utilized as a wearable wellness tool to encourage breathing exercises and mindfulness practices.

Beads and Markers as wearables have been used for generations among multiple cultures. The unique utility invention of Breath Beads addresses a simple tool that is worn (on a person) and when used with a little education functions as a piece of equipment for breathing exercises and aid for meditation practices.

### SUMMARY OF THE INVENTION

The unique utility invention of Breath Beads addresses a simple tool that on a person, often worn but not always worn, and when used with a little education functions as a piece of equipment for breathing exercises and aid for meditation practices. Current models include a bracelet form. Future products could include necklaces, keychains, phone cases, rings, etc. to further the use of beads/markers that function as a breath counter and respiratory timer often associated with meditation practices. Breath Beads is a no-tech, readily available tool that separates it from other meditation practices led through digital platforms. Breath Beads contribute to spiritual wellness, but is available for secular use or any religion or practice which separates it from other rosaries or prayer breads.

### DRAWING DESCRIPTIONS

I/III is a front view of a Breath Beads: 365 Model showing my new design;

II/III is a labeled front view thereof;

III/III is a front view of the Breathful Moments Model

### DETAILED DESCRIPTION OF THE INVENTION

Macey Hickox, have invented Breath Beads: A Wearable Wellness Tool. The tool may be used as a bracelet (tool that is commonly on a person, often worn but not always: products could include necklaces, keychains. phone cases, rings, etc.) and is designed to help tally respirations with education on respiratory rate paced 6 breaths per minute for a specified duration (typically 1-5 minutes). Each bead (or marker) represents one respiration cycle of an inhale and an exhale. Common paces include 5 seconds inhale and 5 seconds exhale or 3 seconds inhale and 7 seconds exhale. Exercises are encouraged to be performed three times a day. Users are encouraged to perform respirations with prompted positive cognitive phrases to optimize healthy brain-body coherence.

### Description of the Breath Beads 365 Model

The design is crafted with a unique Finish bead (marker), five similar beads (markers) followed by another distinct bead (marker) indicating one minute of respirations. These are the three elements of the design. The five similar respiratory+distinct Minute bead (markers) repeats five times around the bracelet which ends at the unique Finish bead (marker). The total number of beads (markers) is 30 to indicate 30 respirations over a five minute duration. To summarize, there is 1 Finish bead (marker), 25 similar beads (markers) and 4 distinct Minute beads (markers) arranged in the specific pattern listed above. Filler beads also known as spacers (markers) may be utilized for style or sizing purposes without impeding the intent of the Breath Beads Models. The main three elements may differ in size, texture, or shape which allow the tool to be used as a tactile device as set forth in the cross referenced Breath Beads Design Patent Application (Cross Reference Design Patent 29/922,090). Breath Meditations are added to the respirations by encouraging a silent phrase to be repeated with the inhalation and with an exhalation.

### Description of the Breathful Moments Model

The Breathful Moments model is an example of a different design, but same utility of using a beaded/marker bracelet to count breaths at a specified respiratory rate often with breath meditation phrases. This model continues with the same resonant breathing respiratory rate of 6 breaths per minute. The Breathful Moments model is a one minute breathing practice. The utility of the bead/marker bracelets could vary in design for any duration.

### Other Breath Beads Designs

The utility of Breath Beads: A Wearable Wellness Tool could be applied to many designs. The tool may be used as a bracelet (tool that is commonly on a person, often worn, but not always: future products could include necklaces. keychains, phone cases, rings, etc.) and is designed to help tally respirations with education on respiratory rate paced 6 breaths per minute for a specified duration (typically 1-5 minutes). The utility can of breath beads could be applied to numerous designs that have variations in respiratory rate or duration of practice.

### Breath Meditations and Breath Prayers

Breath Meditations are added to the respirations by encouraging a silent phrase to be repeated from the heart, with the mind during the inhalation phase and during the exhalation phase. Below is an example of a breath meditation:
“The human body is an incredible machine, but most people only get out of that machine what their mind allows them to.”—Rich Froning
Inhale: “My body is incredible”; Exhale: “I'm a machine.”

When used with Breath Beads, the inhalation phrase, “My body is incredible” would be said silently in mind at a slow breathing rate such as 5 seconds, and the exhalation phrase, “I'm a machine” would be said silently in mind at a slow breathing rate such as 5 seconds. The practice would be continued around the beads. If using the 365 Breath Beads model this would equal 30 repetitions. Users are encouraged to perform any amount of breaths they have time for, even if it's only a one minute practice.

### Science of Breathing and Meditation

Breathing slowly around 6 breaths per minute is known as the resonant or coherent breathing rate that is ideal for our bodies. This also is known to improve our heart health studied in the form of heart rate variability and has a positive effect on the parasympathetic nervous system. Benefits of improving the parasympathetic nervous system balance the body's heightened sympathetic stress response and promotes the rest and digest, heal and feel functions of the body. Adding a positive phrase to the inhalation and exhalation of breathing incorporates further emotional, mental and spiritual benefits of what I call, positive breathing. Having a cued response said repeatedly is a unique utility of breath meditations and using Breath Beads. Most meditation practices ask participants to imagine or picture vague concepts. Breath meditations with the use of breath beads provide specific paced breathing cues and meditation prompts.

### Material of Breath Beads

Current models use beads of various types: natural and synthetic. Examples may include stone, metal, glass, crystal, and wood. Markers made of any bead type including plastic, acrylic, fabric or yarn could also work with Breath Beads utility and design. Current models utilize elastic for the core of the bracelet, but metal, cord, leather, fabric or strings can easily be substituted.

I claim:

1. A wearable wellness tool for respiration counting or timing that may used for meditation or prayer, comprising
a. Beads; or
b. Markers.
